(19) United States
(12) Patent Application Publication
(10) Pub. No.: US 2020/0163863 A1
(43) Pub. Date: May 28, 2020

(54) WATER-RESISTANT COSMETIC MEANS FOR TEMPORARILY CHANGING THE COLOUR OF KERATIN-CONTAINING MATERIALS II

(71) Applicant: Henkel AG & Co. KGAA, Duesseldorf (DE)

(72) Inventors: KONSTANTIN GOUTSIS, Juechen (DE); DANIELA KESSLER-BECKER, Leverkusen (DE)

(73) Assignee: Henkel AG & Co. KGAA, Duesseldorf (DE)

(21) Appl. No.: 16/630,288

(22) PCT Filed: Jul. 2, 2018
(86) PCT No.: PCT/EP2018/067834

§ 371 Date: Jan. 10, 2020

(30) Foreign Application Priority Data
Jul. 11, 2017 (DE) 10 2017 211 852.0

(51) Int. Cl. A61K 8/81; A61K 8/04; A61K 8/29; A61Q 1/10; A61Q 5/06

(52) U.S. Cl. CPC A61K 8/8147; A61K 8/046; A61K 8/042; A61K 8/817; A61K 8/29; A61Q 1/10; A61K 2800/5424; A61Q 5/06; A61K 2800/651; A61K 2800/621; A61K 2800/594; A61K 2800/5428; A61K 2800/43; A61K 8/8188

(57) ABSTRACT

A cosmetic agent and a method for improving water resistance of a pigment-containing cosmetic composition are provided. In an exemplary embodiment, a cosmetic agent comprises (a) at least one anionic polymer A, (b) at least one amphoteric polymer B, and (c) at least one pigment. These cosmetic agents display an extremely good water resistance but may, however, be removed without residue in one application of surfactant-containing cleaning agents.

### CROSS-REFERENCE TO RELATED APPLICATION

This application is a U.S. National-Stage entry under 35 U.S.C. § 371 based on International Application No. PCT/EP2018/067834, filed Jul. 2, 2018, which was published under PCT Article 21(2) and which claims priority to German Application No. 10 2017 21 1852.0, filed Jul. 11, 2017, which are all hereby incorporated in their entirety by reference.

### TECHNICAL FIELD

This application concerns the technical subject of water-resistant cosmetic agents for the temporary color change of materials containing keratin, in particular human skin and/or human hair. The subject of the application is cosmetic agents containing at least one anionic polymer A based on acrylic acid and acrylates, at least one amphoteric polymer B as well as at least one pigment. Finally, the subject of the present application is processes for the temporary coloration of keratin fibers and the skin by applying these agents, and the use of a combination of anionic polymer A and amphoteric polymer B in pigment-containing cosmetic agents to improve the water resistance of these agents.

### BACKGROUND

Change of the color of keratinous surfaces, especially human skin and/or hair, represents an important field in modern cosmetics. Experts know various coloration systems for changing hair color, depending on requirements. Oxidation dyes are used for permanent, intensive colorations with good fastness characteristics and good gray coverage. These dyes usually contain oxidation colorant precursors, so-called developer components and coupler components, which combine with one another to form the actual colorants under the influence of oxidization agents, such as hydrogen peroxide. Oxidization dyes are exemplified by very long-lasting color results.

On application of direct colorants, colorants that are already fully formed diffuse from the dye into the hair fiber. In comparison to oxidative hair coloration, the colorations obtained with direct colorants have lower durability and faster washability. Colorations with direct colorants usually remain on the hair for a period of between about 5 and about 20 hair washes.

Various processes are known in prior art for changing the skin color, in particular for tanning the skin or for reducing unwanted age spots. Tanning the skin can be achieved, for example, by using colorants which penetrate the skin layers and produce a lasting tan. For the reduction of age spots, active substances are applied that penetrate the skin and break up the melanin accumulations that cause the age spots. In this case also, a long-lasting change in skin color, which cannot be removed or can only be removed with difficulty by cleaning the skin, can be achieved.

In modern fashion trends, there is also a demand for color effects that only remain on the hair and/or skin for a short period of time and then may be removed completely without residue from the hair and/or skin by a single wash with surfactant-containing cleaning agents. When in contact with water or sweat, however, the color effect must be retained to stop the color from running under environmental influences such as rain or sweat. Direct colorants diffuse more or less strongly into the hair fiber or the skin surface and last for several washes with surfactant-containing cleaning agents. This class of colorant is therefore not suitable for removal of the color effect without residue by a single wash with surfactant-containing cleaning agents.

The application of color pigments for temporary color changes on the hair and/or the skin is known. Color pigments are generally understood to mean insoluble, color-giving substances These are present, undissolved, in the form of small particles in the color formulation and are only deposited on the outside of the hair fibers and/or the skin surface. Therefore, they can be removed without residue by a single wash with surfactant-containing cleaning agents. Various products of this type are available on the market under the name of hair mascara.

As the removal of hair mascaras can be achieved by washing the hair, they are as a rule designed as “leave-on” products. It is of particular advantage for the users of a “leave-on” product if they can, at the same time as the temporary color change, perform a slight temporary re-shaping of the hair. Temporary re-shaping involves, for example, styles such as curling, straightening, back-combining or even setting. Temporary re-shaping can be achieved by means features example of styling agents such as hair sprays, hair waxes, hair gels, setting lotions, blow-dries, styling sprays etc. Temporary re-shaping is also referred to as hair styling or styling, shaping agents are also referred to as styling agents.

In prior art, products which allow a temporary change in color and/or shape of keratinous surfaces are already known. Those products mostly contain a mixture of film-forming polymers and pigments. However, the products are insufficiently water-resistant.

### BRIEF SUMMARY

A cosmetic agent and a method for improving water resistance of a pigment-containing cosmetic composition are provided. In an exemplary embodiment, a cosmetic agent comprises (a) at least one anionic polymer A comprising at least one structural unit of the formula (I) and at least one structural unit of the formula (II) and at least one structural unit of the formula (III)

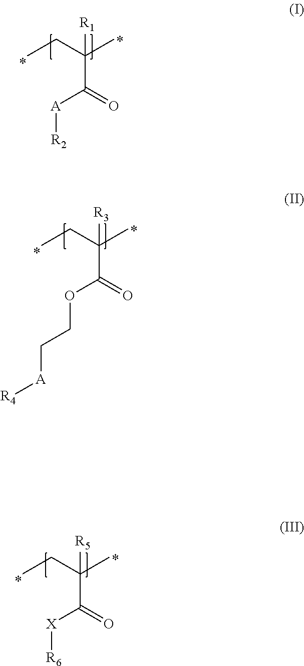

where R₁, R₃ and R₅, independently of each other, stand for hydrogen or a C₁-C₄-alkyl group, R₂ stands for a branched saturated C₆-C₁₂-alkyl group, R₄ stands for a branched saturated C₂-C₅-alkyl group, R₆ stands for hydrogen, a C₁-C₄-alkyl group or a C₂-C₈-hydroxyalkyl group, and X, independently of each other, stand for oxygen, sulfur or an NH group, (b) at least one amphoteric polymer B comprising at least one structural unit of the formula (IV) and at least one structural unit of the formula (V)

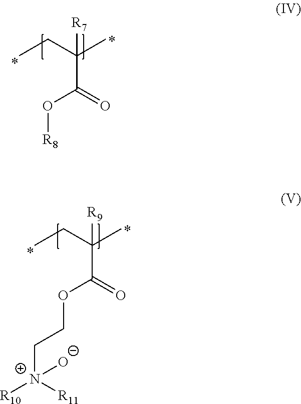

where R₇ and R₉, independently of each other, stand for hydrogen or a C₁-C₄-alkyl group, R₈ stands for a linear saturated C₁-C₃₀-alkyl group, and R₁₀ and R₁₁, independently of each other, stand for a C₁-C₄-alkyl group, and (c) at least one pigment.

In another embodiment, a method for improving water resistance of a pigment-containing cosmetic composition includes adding to the pigment-containing cosmetic composition a cosmetic agent comprising: (a) at least one anionic polymer A, comprising at least one structural unit of the formula (I) and at least one structural unit of the formula (II) and at least one structural unit of the formula (III)

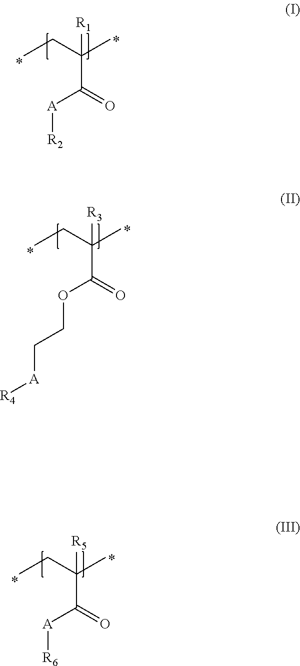

where R₁, R₃ and R₅, independently of each other, stand for hydrogen or a C₁-C₄-alkyl group, R₂ stands for a branched saturated C₆-C₁₂-alkyl group, R₄ stands for a branched saturated C₂-C₅-alkyl group, R₆ stands for hydrogen, a C₁-C₄-alkyl group or a C₂-C₈-hydroxyalkyl group, and A stands for oxygen, sulfur or an NH group, and (b) at least one amphoteric polymer B, comprising at least one structural unit of the formula (IV) and at least one structural unit of the formula (V)

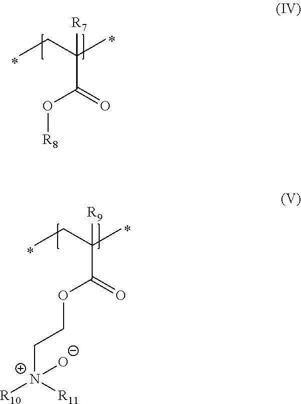

where R₇ and R₉, independently of each other, stand for hydrogen or a C₁-C₄-alkyl group, R₈ stands for a linear saturated C₁-C₃₀-alkyl group, and R₁₀ and R₁₁, independently of each other, stand for a C₁-C₄-alkyl group.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The aim of the present disclosure comprised the provision of a mascara product which can be applied in a variety of ways and which enables the temporary color change of hair and/or the skin. The product was to be adaptable so that it can be applied using a sponge, a brush or a spray application. The color change was to be simple and damage-free and able to be removed from the hair and/or the skin without residue by a single wash with surfactant-containing cleaning agents. Until the next wash, the product on the keratinous surface was to be extremely resistant to outside influences, i.e. there should be no occurrence of color loss or other shedding of the product through the action of water or sweat, abrasion on textiles or combs. At the same time, the color-changed keratinous surface was to have good cosmetic characteristics.

Surprisingly, it has been shown that these aims can be achieved by using color pigments and a combination of anionic polymer A and amphoteric polymer B. The use of this polymer combination leads to high water resistance of the cosmetic agents without, however, any adverse effect on the cosmetic characteristics of these agents. Further, the color can be removed without residue by a single wash with surfactant-containing cleaning agents and, in contrast to coloration with oxidizing colorants and direct colorants or bleaching with oxidizing agents, leads only to a temporary coloration of the keratinous surfaces, particularly the skin and/or hair.

The first subject of the present disclosure is thus a cosmetic agent containing

a) at least one anionic polymer A, comprising at least one structural unit of the formula (I) and at least one structural unit of the formula (II) and at least one structural unit of the formula (III)

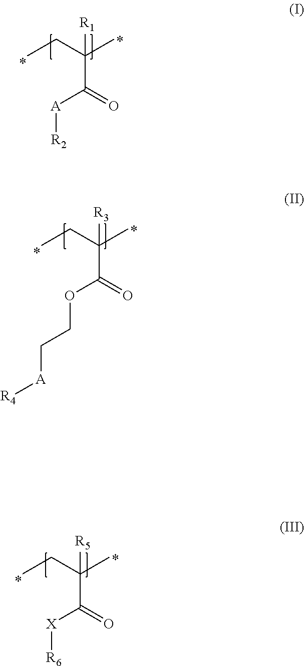

where
R₁, R₃ and R₅, independently of each other, stand for hydrogen or a C₁-C₄-alkyl group,
R₂ stands for a branched saturated C₆-C₁₂-alkyl group,
R₄ stands for a branched saturated C₂-C₅-alkyl group
R₆ stands for hydrogen, a C₁-C₄-alkyl group or a C₂-C₈-hydroxyalkyl group, and
A and X, independently of each other, represent oxygen, sulfur or an NH group,
b) at least one amphoteric polymer B, comprising at least one structural unit of the formula (IV) and at least one structural unit of the formula (V)

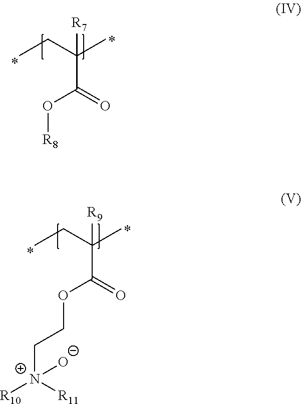

where
R₇ and R₉, independently of each other, stand for hydrogen or a C₁-C₄-alkyl group,
R₈ stands for a linear saturated C₁-C₃₀-alkyl group, and
R₁₀ and R₁₁, independently of each other, stand for a C₁-C₄-alkyl group, and
c) at least one pigment.

According to the formula above and all subsequent formulae a chemical bond, designated by the symbol “*”, stands for the free valence of the corresponding structure fragment. Free valence is to be understood here as the number of atomic bonds that originate from the corresponding structural fragment at the position designated with the symbol “*”. In the present disclosure, an atomic bond preferably originates from the positions of the structural fragments designated with the symbol “*” to further structural fragments.

In the present disclosure, the term “anionic polymers” is understood to mean those polymers which carry at least one structural unit with permanently anionic groups in a protic solvent under standard conditions, whereby the anionic groups have to be compensated for by counterions while maintaining electroneutrality. In the present disclosure, anionic groups include in particular carboxyl groups

In the present disclosure, “amphoteric polymers” are understood to mean those polymers which carry at least one permanently anionic and at least one permanently cationic group in a protic solvent under standard conditions. The permanently anionic and cationic group can be sited within the same structural unit or in different structural units of the polymer. Preferred amphoteric groups are represented by alkylamine oxides.

Save where otherwise stated, the term wt.-% herein refers to the total weight of the present disclosure's cosmetic agents, whereby the sum of all ingredients of the agents of the present disclosure is about 100% by wt.-%. If the cosmetic agents of the present disclosure contain at least one propellant, the term to wt.-% refers to the total weight of the cosmetic product including the said propellant.

As a first essential component a), the cosmetic agent of the present disclosure contains at least one anionic polymer A, comprising at least one structural unit of the formulae (I) to (III). In the structural units of the formulae (I) to (III), the radicals R₁, R₂, R₅ and R₆ can stand for C₁-C₄-alkyl groups. Examples of groups of this type are methyl, ethyl, propyl, isopropyl, butyl, sec-butyl, isobutyl and tert-butyl groups. Further, the radicals R₂ and R₄ in the structural units of the formulae (I) and (II) can stand for branched C₂-C₁₂-alkyl groups. Groups of this type are, for example, tert-butyl, isobutyl and 2,2,4-trimethylpentyl groups. The radical R₆ in the structural formula (III) can stand for a C₂-C₈-hydroxyalkyl group. Groups of this type are, for example, 2-hydroxypropyl, hydroxy butyl and hydroxy pentyl groups.

In the present disclosure it is preferable if the radicals R₁, R₃ and R₅ in formulae (I) to (III) stand for certain groups. Preferred design models of the subject of the present disclosure are therefore exemplified by the fact that in the structural units of the formulae (I) and (II) the radicals R₁ and R₃, in each case independently of each other, stand for a methyl group and that in the structural unit of the formula (III) the radical R₅ stands for hydrogen. Preferably, therefore, anionic polymers based on methacrylates, meth acrylamides, acrylic acid and acrylates are used. The use of this type of anionic polymers in combination with the amphoteric polymer B leads to particularly high water resistance of the cosmetic agents of the present disclosure.

Furthermore, it has been proved to be an advantage in present disclosure if the radical R₂ stands for certain groups in the structural unit of the formula (I). It is therefore preferable that in the structural unit of the formula (I) the radical R₂ stands for a branched saturated C₆-C₁₀-alkyl group and in particular for a *—C(CH₃)₂—CH₂—C(CH₂)₃-group. Here the * symbol indicates the connection of the radical R₂ with group A of the structural unit of the formula (I). The radical R₂ is thus bound to group A of the structural unit of the formula (I) via the CH₂-group.

Furthermore, it is preferable in the present disclosure if A respectively stands for an NH group in the structural units of the formulas (I) and (II). Preferred anionic polymers therefore contain acrylamides and meth acrylamides. The application of anionic polymers A based on acrylamides and meth acrylamides in combination with the amphoteric polymer B leads to improved resistance to outside environmental influences without having an adverse effect on washability with surfactant-containing cleaning agents.

In addition, it has proved to be an advantage in the present disclosure if the group X in the structural unit of the formula (III) represents an oxygen atom It is therefore preferable in the present disclosure if X in the structural unit of the formula (III) stands for an oxygen atom. It is preferable if the anionic polymer A therefore contains acrylic acid and its esters or methacrylic acid and its esters as the structural unit of the formula (III).

The radical R₄ in the structural unit of the formula (II) stands for a branched saturated C₂-C₅-alkyl group. However, it is preferable in the present disclosure if the radical R₄ stands for certain branched saturated C₂-C₅-alkyl groups. Preferred design models of the first subject of the present disclosure are therefore exemplified by the fact that in the structural unit of the formula (II) the radical R₄ stands for a branched saturated C₂-C₅-alkyl group, and in particular for a *—C(CH₃)₃-group. Here the * symbol indicates the connection of the radical R₄ with group A of the structural unit of the formula (II). The radical R₄ is thus bound to group A of the structural unit of the formula (II) via the tertiary carbon atom The application of anionic polymers A, which contain branched alkylamino alkyl acrylates, has proved to be an advantage in connection with the amphoteric polymer B in relation to water resistance and removal without residue by surfactant-containing cleaning agents

It is preferable if the structural units of the formula (III) are acrylic acid or methacrylic acid. It is therefore an advantage in the present disclosure if the radical R₆ in the structural unit of the formula (III) stands for hydrogen.

In addition to the structural units of the formulas (I) to (III) with the above-mentioned significance of the radicals R₁ to R₆, it can be an advantage in the present disclosure if the anionic polymer A contains further structural units based on acrylates and methacrylates The preferred design models of the first subject of the present disclosure are therefore exemplified by the fact that the anionic polymer A additionally has at least one structural unit of the formula (IIIa),

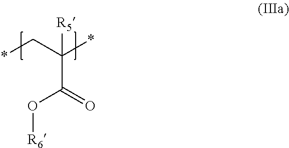

where
R₅′ stands for hydrogen or a methyl group, in particular a methyl group, and
R₆′ stands for a methyl group.

The application of anionic polymers A, which, together with the structural units of the formulas (I) to (III), also contains a further structural unit of the formula (IIIa) based on acrylates and methacrylates, has proved to be particularly advantageous in combination with the minimum one amphoteric polymer B in relation to the water resistance of the cosmetic agents, but without adversely influencing the ability to achieve complete removal by surfactant-containing cleaning agents.

Further, it can be preferable in the present disclosure if the anionic polymer A, in addition to the structural units of the formulas (I) to (III) with the above-mentioned radicals R₁ to R₆, also contains a structural unit (IIIb) based on 2-hydroxypropyl acrylates and/or 2-hydroxypropyl methacrylates. It is therefore particularly preferable in the present disclosure if the anionic polymer A additionally has at least one structural unit of the formula (IIIb)

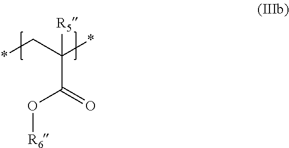

where
R₅″ stands for hydrogen or a methyl group, in particular a methyl group, and
R₆″ represents a 2-hydroxypropyl group.

The application of anionic polymers A, which, in addition to the structural units of the formulas (I) to (III) and (IIIa), also contains a further structural unit of the formula (IIIb) based on 2-hydroxypropyl acrylates and/or 2-hydroxypropyl methacrylates, has been found in combination with the at least one amphoteric polymer B to be particularly advantageous in relation to the water resistance of the cosmetic agents, but without adversely affecting the ability to achieve complete removal with surfactant-containing cleaning agents.

In this context, anionic polymers A which contain the structural units (I) to (III) and (IIIa) and (IIIb) are particularly preferred. In the present disclosure, it is therefore particularly of advantage to use anionic polymers A which contain at least one structural unit of the formula (I), at least one structural unit of the formula (II), at least one structural unit of the formula (III), at least one structural unit of the formula (IIIa) and at least one structural unit of formula (IIIb)

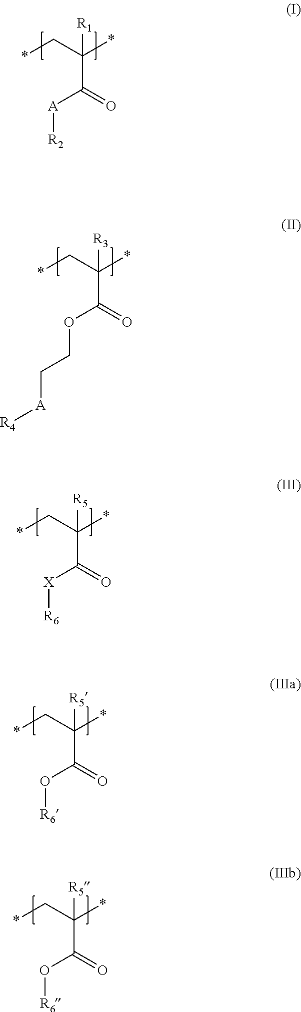

where
R₁ and R₃, each stand for a methyl group,
R₅ and R₆, independently of each other, stand for hydrogen,
R₅′ and R₅″, independently of each other stand for hydrogen or a methyl group, in particular a methyl group,
R₂ stands for a *—C(CH₃)₂—CH₂—C(CH₂)₃-group,
R₄ stands for a *—C(CH₃)₃-group,
A stands for an NH group,
X stands for oxygen,
R₆′ stands for a methyl group and
R₆″ stands for a 2-hydroxypropyl group.

The application of this type of anionic polymers A in combination with the amphoteric polymer B has proved to be of particular advantage with regard to resistance to outside environmental influences on the one hand and the washability of the cosmetic agents in the present disclosure by surfactant-containing cleaning agents on the other hand. The application of such anionic polymers A in combination with amphoteric polymers B in pigment-containing cosmetic agents therefore results in a water-resistant temporary coloration of keratinous surfaces, which can be removed by a single application of surfactant-containing cleaning agents. In addition, this polymer combination can achieve a high and flexible hairstyle hold that does not become sticky when exposed to moisture, thereby making an unnatural impression.

The preferred cosmetic agents in the present disclosure contain the minimum one anionic polymer A in a total amount of from about 0.1 to about 10 wt.-%, preferably from about 0.5 to about 8.0 wt.-%, preferably from about 1.0 to about 5.5 wt.-%, particularly preferred from about 1.0 to about 2.5 wt.-%, in relation to the total weight of the cosmetic agent. The use of these amounts of the anionic polymer A in combination with the amphoteric polymer B results in a high resistance to outside environmental influences, but without adversely affecting the applicability of these agents and their stability in storage. When these amounts of polymer are applied in agents for the simultaneous temporary change in color and shape of the hair, in addition to water-resistant coloring, it is also possible to retain a high and flexible hold for the hairstyle.

As a second essential component b), the cosmetic agent in the present disclosure contains at least one amphoteric polymer B which contains at least one structural unit of the formulae (IV) and (V). In the structural unit of the formula (IV), the radical R₈ can stand for a linear C₁-C₃₀-alkyl group. Examples of such groups are methyl, ethyl, propyl, butyl, pentyl, hexyl, heptyl, octyl, nonyl, decyl, capryl, caprine, lauryl, myristic and stearyl groups.

In the present disclosure, it is preferable if the radicals R₇ and R₉ in the formulae (IV) and (V) stand for certain groups. Preferred design models of the first subject of the present disclosure are therefore exemplified by the fact that in the structural units of the formulas (IV) and (V) the radicals R₇ and R₉, independently of each other, stand for a methyl group. Amphoteric polymers B based on methacrylates and methacrylamine oxides are therefore preferably applied The use of this type of amphoteric polymers B in combination with the above-mentioned anionic polymers A leads to particularly high water resistance of the cosmetic agents in the present disclosure.

Furthermore, it has proved to be an advantage in the present disclosure if the radical R₈ in the structural unit of the formula (IV) stands for certain groups. It is therefore preferable that in the structural unit of the formula (IV), the radical R₈ represents a methyl group. The application of amphoteric polymers B, which contain methyl methacrylate, has proved to be a particular advantage in connection with the preferred anionic polymers A with regard to water resistance on the one hand and the ability to wash out the pigments with surfactant-containing cleaning agents on the other hand.

Furthermore, it is preferable in the present disclosure if the radicals R₁₀ and R₁₁, independently of each other, stand for a methyl group. The preferred amphoteric polymers B therefore contain at least one structural unit based on methacrylamine oxides. The application of amphoteric polymers B based on methacrylamine oxides in combination with the above-mentioned anionic polymers A leads to improved resistance to outside environmental influences, without, however, adversely affecting the ability to be washed out with surfactant-containing cleaning agents.

In addition to the structural units of the formulas (IV) and (V) with the above-mentioned significance of the radicals R₇ to R₁₁ in the present disclosure it can be of advantage if the amphoteric polymer B contains further structural units based on meth acrylates. It is therefore preferable in the present disclosure if the amphoteric polymer B additionally has at least one structural unit of the formula (IVa),

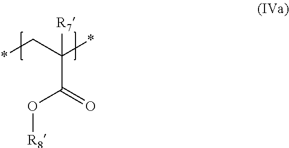

where
R₇′ stands for a methyl group and
R₈′ stands for a linear saturated C₁₈-alkyl group, contains. The application of amphoteric polymers B, which, in addition to the structural units of the formulas (IV) and (V), also contains a further structural unit of the formula (IVa) based on stearyl meth acrylate, has proved to be of particular advantage in combination with the minimum one above-mentioned anionic polymer A in relation to the water resistance of the cosmetic agents, but without adversely affecting the ability to effect complete removal with surfactant-containing cleaning agents.

Therefore in the present disclosure, those amphoteric polymers B are put to particularly advantageously use which contain at least one structural unit of the formula (IV), at least one structural unit of the formula (IVa) and at least one structural unit of the formula (V)

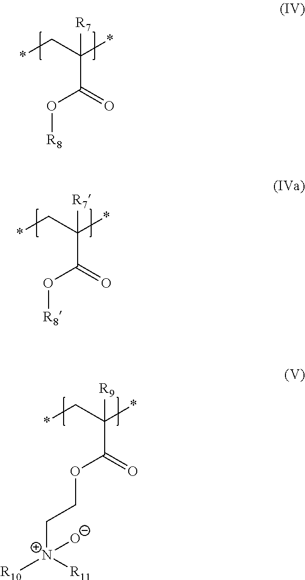

where
R₇, R₇′ and R₈, independently of each other, stand for a methyl group,
R₈ stands for a methyl group,
R₈′ stands for a linear saturated C 18-alkyl group and
R₁₀ and R₁₁, independently of each other, stand for a methyl group.
The application of this type of amphoteric polymers B based on methyl meth acrylate, stearyl meth acrylate and ethylamine oxide meth acrylate in combination with the above-mentioned anionic polymers A has proved to be of particular advantage in relation to resistance to outside environmental influences on the one hand and the washability of the cosmetic agents in the present disclosure by surfactant-containing cleaning agents on the other hand. Therefore, the application of this type of amphoteric polymers B in combination with the above-mentioned anionic polymers A in pigment-containing cosmetic agents results in the water-resistant temporary coloration of keratinous surfaces, which can be removed by a single application of surfactant-containing cleaning agents. In addition, this polymer combination can achieve a high and flexible hairstyle hold that does not become sticky when exposed to moisture, thereby making an unnatural impression.

The preferred cosmetic agents in the present disclosure contain the minimum one amphoteric polymer B in a total amount of from about 0.1 to about 10% by weight.-%, preferably from about 0.5 to about 8.0 wt.-%, preferably from about 1.0 to about 5.5 wt.-%, in particular from about 1.0 to about 2.0 wt.-%, in relation to the total weight of the cosmetic product. The use of these amounts of the amphoteric polymer B, in combination with the anionic polymer A, results in a high resistance to external environmental influences, without, however, adversely affecting the applicability of these agents and the storage stability. When these amounts of polymer are used in agents for the simultaneous temporary change in color and shape of the hair, in addition to a water-resistant coloring, a high and flexible hold of the hairstyle is also made possible. The use of these amounts of the amphoteric polymer B, in combination with the anionic polymer A, results in high resistance to outside environmental influences, without, however, adversely affecting the applicability of these agents and their stability in storage. When these amounts of polymers are used in agents for the simultaneous temporary change in color and shape of the hair, in addition to water-resistant coloration, it is also possible to achieve a high and flexible hold of the hairstyle.

In the present disclosure, it has proved to be of particular advantage if the anionic polymer A and the amphoteric polymer B are applied in certain weight ratios. The preferred design models of the first subject of the present disclosure are therefore exemplified by the fact that the cosmetic agent has a weight ratio of the at least one anionic polymer A to the at least one amphoteric polymer B of from about 4:1 to about 1:1, preferably of from about 3:1 to about 1:1, preferably of from about 2:1 to about 1:1, in particular about 1:0.75. The above-mentioned weight ratio refers to the total amounts of polymer A and polymer B. If a mixture of different polymers A and/or B is therefore used, the total amount of the mixture of different polymers A and/or the mixture of different polymers B used in order to calculate the weight ratio. By using the above-mentioned weight ratios of anionic polymer A to amphoteric polymer B, the resistance to outside environmental influences, in particular the water resistance, can be significantly increased without, however, adversely affecting the removal without residue by surfactant-containing cleaning agents. If the cosmetic agents are styling agents for keratinous fibers, this polymer combination not only achieves high water resistance for the coloring, but also a high, flexible and moisture-resistant hairstyle hold.

As a third essential component c), the cosmetic agent in the present disclosure contains at least one pigment. Pigments in the sense of the present disclosure are understood to mean coloring compounds which have a solubility of less than about 0.1 g/l in water at about 20° C. Water solubility can be demonstrated, for example, using the method described below: about 0.1 g of the pigment is weighed out in a beaker. about 0.1 g of the pigment is weighed out in a beaker. A stir bar is added. It is then made up to about 11 with distilled water (about 20° C.). It is stirred for an hour. If undissolved constituents of the pigment are still visible in the mixture after this period, the solubility of the pigment is below about 0.1 g/l.

The cosmetic agents in the present disclosure should preferably lead to a temporary color in the form of “metallic” effects. White pigments do not fall under the definition of the color pigment. White pigments are achromatic inorganic pigments with a high refractive index (usually greater than about 1.8), which are usually made synthetically and above all to produce optical whiteness in paints or as a filler, e.g. in plastics. White pigments such as titanium dioxide or zinc dioxide are therefore expressly not included in the definition of the color pigment.

The color pigments are present in the agents in the form of small undissolved particles which do not diffuse into the keratinous surface, but which accumulate in the polymer film formed by component b) on the outside of the keratinous surface.

Suitable color pigments may be organic or inorganic in origin. The preferred color pigments are selected from synthetic or natural inorganic pigments. In organic color pigments of natural origin may, for example, be produced from chalk, ocher, umber, green earth, burnt terra di Siena or graphite. Furthermore black pigments such as iron oxide black, colored pigments such as ultramarine or iron oxide red and fluorescent or phosphorescent pigments can be used as inorganic color pigments.

The preferred design models of the first subject of the present disclosure are therefore exemplified by the fact that the cosmetic agent contains as color pigment (b) at least one inorganic color pigment which is selected from (i) colored metal oxides, (ii) metal hydroxides, (iii) metal oxide hydrates, (iv) silicates, (v) metal sulfides, (vi) complex metal cyanides, (vii) metal sulfates, (viii) bronze pigments and/or from (ix) colored pigments based on mica or mica, coated with at least one metal oxide and/or a metal oxychloride, and (x) mixtures thereof.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Black iron oxide (C.I. 77499), yellow iron oxide (C.I. 77492), red and brown iron oxide (C.I. 77491), manganese violet (C.I. 77742), ultramarine (sodium aluminum sulfa-silicate, C.I. 77007, Pigment Blue 29), chromium oxide hydrate (C.I. 77289), Iron Blue (Ferric ferrocyanide, C.I. 77510) and carmine (cochineal), are all particularly preferable pigments.

Color pigments which are particularly preferred in the present disclosure are colored pearlescent pigments. These are usually based on mica and/or mica and can be coated with one or more metal oxides. Mica is a layered silicate. The most important examples of these silicates are muscovite, phlogopite, paragonite, biotite, lepidolite and margarite. To produce the pearlescent pigments in conjunction with metal oxides, the mica, predominantly muscovite or phlogopite, is coated with a metal oxide.

As an alternative to natural mica, synthetic mica coated with one or more metal oxides can also be used as a pearlescent pigment. Particularly preferable pearlescent pigments are based on natural or synthetic mica (mica) and are coated with one or more of the aforementioned metal oxides. The color of the given pigments can be varied by varying the thickness of the metal oxide (s) layer.

It is therefore particularly preferable in the present disclosure if the minimum one pigment is a colored pigment based on mica or mica which is coated with one or more metal oxides from the group including titanium dioxide (CI 77891), black iron oxide (CI 77499) and yellow iron oxide (CI 77492), red and/or brown iron oxide (CI 77491, CI 77499), manganese violet (CI 77742), ultramarine (sodium aluminum sulfasilicate, CI 77007, Pigment Blue 29), chromium oxide hydrate (CI 77289), chromium oxide (CI 77288) and/or Iron blue (Ferric Ferrocyanide, CI 77510).

Examples of particularly suitable color pigments are commercially available, for example, under the trade names Rona®, Colorona®, Xirona®, Dichrona® and Timiron® from Merck, Ariabel® and Unipure® from Sensient, Prestige® from Eckart Cosmetic Colors and Sunshine® available from Sunstar.

Color pigments with the trade name Colorona® that are particularly preferable are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferable color pigments with the trade name Xirona® are, for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferable color pigments with the trade name Unipure® are, for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

The color pigments named above whose surface has been hydrophobized are also particularly suitable for some forms of use. It has been found that these pigments can sometimes be of advantage, particularly with storage under extreme conditions.

Silicones, for example, are suitable as water repellents.
Color pigments treated with hydrophobic surfaces are described, for example, in DE 102009051171, the content of which is expressly referred to here.

Because of their excellent light, weather and/or temperature resistance, the use of the above-named inorganic color pigments in the agent in the present disclosure is particularly preferable. It is further preferred if the pigments used have a certain particle size. This particle size leads on the one hand to an even distribution of the pigments in the polymer film formed and on the other hand avoids a rough feeling on the hair or skin after the application of the cosmetic agent. It is therefore of advantage in the present disclosure if the minimum one pigment has an average particle size D 50 from about 1.0 to about 50 μm, preferably from about 5.0 to about 45 μm, preferably from about 10 to about 40 μm, in particular from about 14 to about 30 μm. The average particle size D₅₀ can be determined, for example, using dynamic light scattering (DLS).

Depending on the desired color change on the keratinous surface, the minimum one color pigment c) can be used in different amounts. The more color pigment is used, the greater the extent of the color change. From a certain amount of use, however, the adhesion of the pigments to the keratinous fiber runs against a limit, above which it is no longer possible to increase the extent of the color change by further increasing the amount of pigment used. The more color pigment is applied, the greater the color change in general. Above a certain amount of application, however, the adhesion of the pigments to the keratinous fiber encounters a limit, above which it is no longer possible to increase the extent of the color change by further increasing the amount of pigment applied.

In this context, it has been shown that, on application of the polymer combination in the present disclosure—in particular the preferred and particularly preferred examples mentioned—, a film can be formed on the keratinous surface, which allows the pigments to be held on the surface in particularly large amounts. Particularly preferable design models of the first subject of the present disclosure are exemplified by the fact that the minimum one pigment is contained in a total amount of from about 1.0 to about 25.0 wt.-%, preferably from about 5.0 to about 20.0 wt.-%, preferably from about 7.0 to about 18.0 wt.-%, in particular from about 8.5 to about 15.5 wt.-%, respectively relating to the total weight of the cosmetic agent.

A particularly preferable design model for the first subject matter of the present disclosure is therefore cosmetic agents for the temporary alteration of keratinous fibers containing

a) at least one anionic polymer A, comprising at least one structural unit of the formula (I) and at least one structural unit of the formula (II) and at least one structural unit of the formula (III) and at least one structural unit of the formula (IIIa) and at least one structural unit of the formula (IIIb)

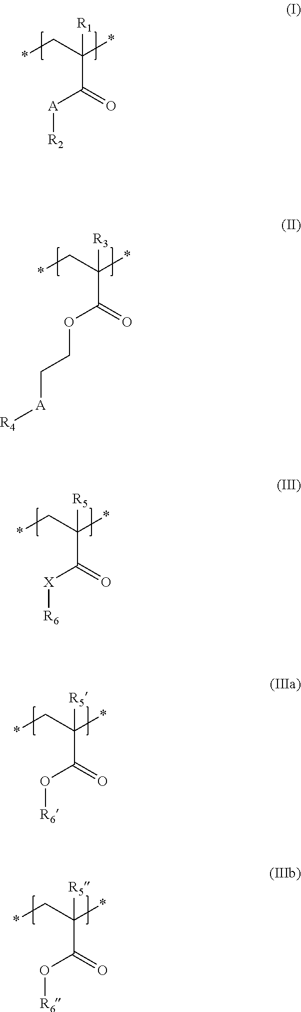

where
R₁ and R₃ each stand for a methyl group,
R₅ and R₆, independently each other, stand for hydrogen,
R₅′ and R₅″, independently of each other stand for hydrogen or a methyl group, in particular a methyl group,
R₂ stands for a *—C(CH₃)₂—CH₂—C(CH₂)₃-group,
R₄ stands for a *—C(CH₃)₃-group,
A stands for an NH group,
X stands for oxygen,
R₆′ stands for a methyl group and
R₆″ stands for a 2-hydroxypropyl group,
b) at least one amphoteric polymer comprising at least one structural unit of the formula (IV), at least one structural unit of the formula (IVa) and at least one structural unit of the formula (V)

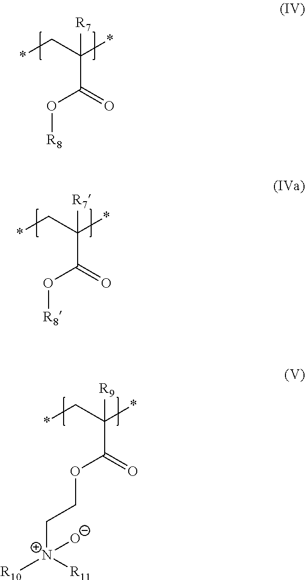

where
R₇, R₇′ and R₈, independently of each other, stand for a methyl group,
R₈ stands for a methyl group,
R₈′ stands for a linear saturated C 18-alkyl group and
R₁₀ and R₁₁, independently of each other, stand for a methyl group and
c) at least one pigment selected from colored pigments based on mica or mica, which are coated with one or more metal oxides from the group including titanium dioxide (CI 77891), black iron oxide (CI 77499), yellow iron oxide (CI 77492), red and/or brown iron oxide (CI 77491, CI 77499), manganese violet (CI 77742), Ultramarine (sodium aluminum sulfasilicate, CI 77007, Pigment Blue 29), chromium oxide hydrate (CI 77289), chromium oxide (CI 77288) and/or iron blue (Ferric Ferrocyanide, CI 77510).
Cosmetic agents of this type have high resistance to outside environmental influences such as rain, sweat, abrasion by textiles and combs but can nonetheless be removed without residue by a single application of surfactant-containing cleaning agents. When applied to keratinous fibers, especially hair, an excellent degree of hold and high moisture resistance are also achieved. The high moisture resistance prevents the hair from becoming sticky when exposed to moisture. When applied to the skin, these agents have good cosmetic properties and do not lead to dry or oily skin.

The agents in the present disclosure contain all essential components in one carrier. In particular, aqueous, aqueous-alcoholic and alcoholic carriers can be used. Alcoholic carriers are particularly preferable. Such carriers contain at least one aliphatic and/or aromatic alcohol with 2 to 8 carbon atoms in a total amount of from about 40 to about 98 wt.-%, preferably from about 50 to about 95 wt.-%, preferably from about 60 to about 90 wt.-%, in particular from about 70 to about 90 wt.-%, respectively in relation to the total weight of the cosmetic agent.

It is of advantage in the present disclosure if certain aliphatic and/or aromatic alcohols are used as carriers. In this context it is therefore preferred if the aliphatic and/or aromatic alcohol with 2 to 8 carbon atoms is selected from the group ethanol, isopropanol, n-propanol, butanol, n-pentanol, 1,2-propanediol, 1,3-propanediol, 1,3-butanediol, 1,4-butanediol, 1,2-hexanediol, 1,6-hexanediol, glycerin, benzyl alcohol, phenoxyethanol, phenylethyl alcohol and mixtures thereof.

Ethanol is particularly preferable as the alcoholic carrier. It has been shown that the use of ethanol leads to particularly good film formation and thus to a particularly high adhesion of the pigments on the keratinous surface. In this case, the color result is particularly even and high resistance to outside environmental influences such as rain, sweat and abrasion is achieved. it is therefore to be preferred in the present disclosure if ethanol is contained in a total amount of from about 40 to about 98 wt.-%, preferably from about 50 to about 95 wt.-%, preferably from about 60 to about 90 wt.-%, in particular from about 70 to about 90 wt.-%, respectively relating to the total weight of the cosmetic agent. The properties described above can also be further improved if a further polyvalent alcohol with lower volatility, for example 1,2-propanediol or glycerol, is added to the ethanol. In this context, it is therefore to be preferred if additionally 1,2-propanediol and /or glycerin is contained in a total amount of from about 0.1 to about 7.0 wt.-%, preferably from about 0.5 to about 5.5 wt.-%, preferably from about 1.0 to about 3.5 wt.-%, in particular of from about 1.5 wt.-% to about 2.5 wt.-%, respectively in relation to the total weight of the cosmetic agent.

The deposit of the pigments on the keratinous surfaces and the film formation of the anionic polymer A and amphoteric polymer B can also be influenced by the water content of the carrier. If the water content is too high, there is a risk that the product will not dry fast enough. In particular, if the agents are set to a lower viscosity (because they are to be sprayed, for example), the color result may be uneven. It is therefore preferred as contemplated herein if the cosmetic agent contains water in a total amount of from 0 to about 30% by weight.-%, preferably from 0 to about 20 wt.-%, preferably from 0 to about 10 wt.-%, in particular from 0 to about 2.0 wt.-%, in relation to the total weight of the cosmetic product. If the water content is too high, there is a danger that the product will not dry fast enough. In particular, if the agents are set to a lower viscosity (because they are to be sprayed, for example), the color result may be uneven. It is therefore preferable in the present disclosure if the cosmetic agent contains water in a total amount of 0 to about 30% wt.-%, preferably from 0 to about 20 wt.-%, preferably from 0 to about 10 wt.-%, in particular from 0 to about 2.0 wt.-%, respectively in relation to the total weight of the cosmetic agent.

The cosmetic agents in the present disclosure can be used for temporary change in color and shape of keratinous fibers at the same time, in particular hair. These agents can also be used to change the color of the skin temporarily. To this end, they can be assembled in the usual forms, for example as a gel, spray, foam or wax. Assembly as a spray is preferred. Sprays of this type can be in the form of aerosols and non-aerosols and can be sprayed from containers familiar to experts. If the agents are assembled as aerosols, at least one propellant is also included. Propellants suitable to the present disclosure may be selected, for example, from N₂O, dimethyl ether, CO₂, air, alkanes with 3 to 5 carbon atoms, such as propane, n-butane, isobutane, n-pentane and isopentane, and mixtures thereof. Dimethyl ether is most particularly preferable used as the propellant. However, the present disclosure expressly also includes the use of propellants of the chlorofluorocarbon type, in particular the fluorocarbons. These propellants are preferably contained in a total amount of from about 30 to about 70 wt.-%, preferably from about 35 to about 75 wt.-%, in particular from about 40 to about 60 wt.-%, respectively in relation to the total weight of the cosmetic agent.

The mascara products known in prior art generally contain fatty substances. These fatty substances can also form a film on the keratinous surfaces, which protects the pigments after application from abrasion or washing away by water. The essential disadvantage of the fatty substances, however, is that they produce a sub-optimal feel on the keratinous surface, in particular a feeling of hardness and a greasy feeling. The keratin surface seems weighted down and also makes a visually greasy impression.

It is therefore to be preferred in the present disclosure if the cosmetic agents only contain a small proportion of fatty substances. The preferred design models of the first subject of the present disclosure are therefore exemplified by the fact that the cosmetic agent contains fatty substances in a total amount of from 0 to about 2.5% wt.-%, preferably from 0 to about 1.5 wt.-%, preferably from 0 to about 0.5 wt.-%, more preferably from 0 to about 0.1 wt.-%, in particular of 0 wt.-%, respectively in relation to the total weight of the cosmetic agent. For the purposes of the present disclosure, “fatty substances” are understood as organic compounds with a solubility in water at room temperature (about 22° C.) and atmospheric pressure (about 760 mm Hg) of less than about 1 wt.-%, preferably less than about 0.1 wt. %. The definition of fatty components expressly only includes uncharged (i.e. non-ionic) compounds. Charged compounds such as fatty acids and their salts are not understood to be a fatty component. Fatty substances in the sense of the present disclosure have at least one saturated or unsaturated alkyl group with at least 12 carbon atoms. If the fatty substances contain an unsaturated alkyl group, this can have one or more double bonds. The molecular weight of the fatty components is a maximum of about 5000 g/mol, preferably a maximum of about 2500 g/mol and particularly preferably a maximum of about 1000 g/mol. The fatty components are neither polyoxyalkylated nor polyglycerylated compounds. Therefore, fatty alcohols or fatty acids which are esterified or etherified with at least two oxyalkyl groups or with at least two glycerol units do not fall under the definition of the fatty substances.

Fatty substances, which are preferably contained in a total amount of about 2.5 wt.-% at most, are selected from the group of (i) C₁₂-C₃₀-fatty alcohols; (ii) C₁₂-C₃₀-fatty acid triglycerides; (iii) diester of one equivalent of ethylene glycol (1,2-ethanediol) with two equivalents of fatty acid (ethylene glycol difatty acid ester); (iv) waxes; (v) hydrocarbons with at least 12 carbon atoms; and (vi) mixtures thereof. C₁₂-C₃₀-fatty alcohols are saturated, mono- or polyunsaturated, linear or branched fatty alcohols with 12 to 30 C atoms. Examples of this type of C₁₂-C₃₀-fatty alcohols are dodecan-1-ol (dodecyl alcohol, lauryl alcohol), tetradecane-1-ol (tetradecyl alcohol, myristic alcohol), hexadecane-1-ol (hexadecyl alcohol, cetyl alcohol, palmityl alcohol), octadecane-1-ol (octadecyl alcohol, stearyl alcohol), arachyl alcohol (eicosan-1-ol), heneicosyl alcohol (heneicosan-1-ol) and/or behenyl alcohol (docosan-1-ol). Examples of branched fatty alcohols are 2-octyl-dodecanol, 2-hexyl-dodecanol and/or 2-butyl-dodecanol. C₁₂-C₃₀-fatty acid triglycerides are understood as mono-, di- and triesters of the trivalent alcohol glycerol with three equivalents of fatty acid. Both structurally identical and different fatty acids within a triglyceride molecule can be involved in the ester formation. Waxes are understood to be the esters of C₁₂-C₃₀-fatty acids with C₁₂-C₃₀-fatty alcohols. As contemplated herein, hydrocarbons are understood to be compounds which consist exclusively of the atoms carbon and hydrogen. Examples of hydrocarbons are mineral oils, liquid paraffin oils (e.g. Paraffinum Liquidum or Paraffinum Perliquidum), isoparaffin oils, semi-solid paraffin oils, paraffin waxes, hard paraffin (Paraffinum Solidum), petroleum jelly and polydecenes. Silicones are not included in the definition of fatty substances.

As contemplated herein, the preferred fatty substances which are not [present] or are contained in the ranges mentioned above are therefore selected from the group of C₁₂-C₃₀-fatty alcohols, the C₁₂-C₃₀-fatty acid triglycerides, C₁₂-C₃₀-fatty acid diglycerides, the C₁₂-C₃₀-fatty acid monoglycerides, the ethylene glycol difatty acid ester, the waxes, the hydrocarbons and mixtures thereof.

The following tables list particularly preferred design models AF 1 to AF 80 of the cosmetic agents as contemplated herein (all details in wt.-%).

| | AF 1 | AF 2 | AF 3 | AF 4 |
|---|---|---|---|---|
| Anionic polymer A ¹⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 5 | AF 6 | AF 7 | AF 8 |
| Anionic polymer A ⁴⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 9 | AF 10 | AF 11 | AF 12 |
| Anionic polymer A ⁵⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 13 | AF 14 | AF 15 | AF 16 |
| Anionic polymer A ¹⁾ | 0.1 to 10 | 0.5 to 8.0 | 1.0 to 5.5 | 1.0 to 2.5 |
| Amphoteric polymer B ⁶⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 17 | AF 18 | AF 19 | AF 20 |
| Anionic polymer A ¹⁾ | 0.1 to 10 | 0.5 to 8.0 | 1.0 to 5.5 | 1.0 to 2.5 |
| Amphoteric polymer B ⁷⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 21 | AF 22 | AF 23 | AF 24 |
| Anionic polymer A ¹⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 25 | AF 26 | AF 27 | AF 28 |
| Anionic polymer A ⁴⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 29 | AF 30 | AF 31 | AF 32 |
| Anionic polymer A ⁵⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 33 | AF 34 | AF 35 | AF 36 |
| Anionic polymer A ¹⁾ | 0.1 to 10 | 0.5 to 8.0 | 1.0 to 5.5 | 1.0 to 2.5 |
| Amphoteric polymer B ⁶⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 37 | AF 38 | AF 39 | AF 40 |
| Anionic polymer A ¹⁾ | 0.1 to 10 | 0.5 to 8.0 | 1.0 to 5.5 | 1.0 to 2.5 |
| Amphoteric polymer B ⁷⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ³⁾ | Add 100 | Add 100 | Add 100 | Add 100 |
| | AF 41 | AF 42 | AF 43 | AF 44 |
| Anionic polymer A ¹⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 45 | AF 46 | AF 47 | AF 48 |
| Anionic Polymer A ⁴⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 49 | AF 50 | AF 51 | AF 52 |
| Anionic polymer A ⁵⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 53 | AF 54 | AF 55 | AF 56 |
| Anionic polymer A ¹⁾ | 0.1 to 10 | 0.5 to 8.0 | 1.0 to 5.5 | 1.0 to 2.5 |
| Amphoteric polymer B ⁶⁾ | 0.1 to 10 | 0.5 to 8.0 | 1.0 to 5.5 | 1.0 to 2.0 |
| Pigment c) | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 57 | AF 58 | AF 59 | AF 60 |
| Anionic polymer A ¹⁾ | 0.1 to 10 | 0.5 to 8.0 | 1.0 to 5.5 | 1.0 to 2.5 |
| Amphoteric polymer B ⁷⁾ | 0.1 to 10 | 0.5 to 8.0 | 1.0 to 5.5 | 1.0 to 20 |
| Pigment c) | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 61 | AF 62 | AF 63 | AF 64 |
| Anionic polymer A ¹⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 65 | AF 66 | AF 67 | AF 68 |
| Anionic polymer A ⁴⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 69 | AF 70 | AF 71 | AF 72 |
| Anionic polymer A ⁵⁾ | 0.1 to 10 | 0.5 to 8.0 | 1.0 to 5.5 | 1.0 to 2.5 |
| Amphoteric polymer B ²⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 73 | AF 74 | AF 75 | AF 76 |
| Anionic polymer A ¹⁾ | 0.1 to 10 | 0.5 to 8.0 | 1.0 to 5.5 | 1.0 to 2.5 |
| Amphoteric polymer B ⁶⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ⁹⁾ | Add 100 | Add 100 | Add 100 | Add 100 |
| | AF 77 | AF 78 | AF 79 | AF 80 |
| Anionic polymer A ¹⁾ | 0.1 to 10 | 0.5 to 8.0 | 1.0 to 5.5 | 1.0 to 2.5 |
| Amphoteric polymer B ⁷⁾ | 0.1 to 10 | 0.5 to 8.0 | 1.0 to 5.5 | 1.0 to 2.0 |
| Pigment c) ⁸⁾ | 1.0 to 25 | 5.0 to 20 | 7.0 to 18 | 8.5 to 15.5 |
| Cosmetic carrier ⁹⁾ | Add 100 | Add 100 | Add 100 | Add 100 |

¹⁾ containing structural units of the formulae (I) to (III) with R₁, R₃ and R₅ each = H or C₁-C₄-alkyl group, R₂ = branched saturated C₆-C₁₂-alkyl group, R₄ = branched saturated C₂-C₅-alkyl group, R₆ = hydrogen, C₁-C₄-alkyl group or C₂-C₈-hydroxyalkyl group, A and X each = O, S or NH,
²⁾ containing structural units of the formulae (IV) and (V) with R₇ and R₉ each = H or C₁-C₄-alkyl group, R₈ = linear saturated C₁-C₃₀-alkyl group, R₁₀ and R₁₁ each = C₁-C₄-alkyl group,
³⁾ aqueous, aqueous-alcoholic or alcoholic carrier,
⁴⁾ containing structural units of the formulae (I) to (III) with R₁ and R₃ each = methyl group, R₂ = *—C(CH₃)₂—CH₂—C(CH₂)₃-Group, R₄ = *—C(CH₃)₃-Group, R₅ and R₆ each = H, A = NH and X = O,
⁵⁾ containing structural units of the formulae (I), (II), (III), (IIIa) and (IIIb) with R₁ and R₃ each = methyl group, R₂ = *—C(CH₃)₂—CH₂—C(CH₂)₃-group, , R₄ = *—C(CH₃)₃-group, R₅ and R₆ each = H, A = NH, X = O, R₅′ and R₅″ each = methyl group, R₆′ = methyl group, R₆″ = 2-hydroxypropyl group,
⁶⁾ containing structural units of the formulae (IV) and (V) with R₇ and R₉ each = methyl group, R₈ = methyl group, R₁₀ and R₁₁ each = methyl group,
⁷⁾ containing structural units of the formulae (IV), (IVa) and (V) with R₇, R₇′ and R₉ each = methyl group, R₈ = Methyl group, R₈′ = linear saturated C₁₈-alkyl group, R₁₀ and R₁₁ each = methyl group,
⁸⁾ selected from colored pigments based on mica or mica, which are coated with one or more metal oxides from the group including titanium dioxide (CI 77891), black iron oxide (CI 77499), yellow iron oxide (CI 77492), red and/or brown iron oxide (CI 77491 , CI 77499), manganese violet (CI 77742), ultramarine (sodium aluminum sulfasilicate, CI 77007, Pigment Blue 29), chromium oxide hydrate (CI 77289), chromium oxide (CI 77288) and/or iron blue (Ferric Ferrocyanide, CI 77510) are coated,
⁹⁾ containing - respectively in relation to the total weight of the respective design model - 70 to 90 wt.-% ethanol and from about 1.5 wt.-% to about 2.5 wt.-% 1,2-propanediol and/or glycerin.

The design models AF 1 to AF80 contain 0 wt.-% fatty substances selected from the group of C₁₂-C₃₀-fatty alcohols, the C₁₂-C₃₀-fatty acid triglycerides, C₁₂-C₃₀-fatty acid diglycerides, the C₁₂-C₃₀-fatty acid monoglycerides, the ethylene glycol difatty acid ester, the waxes, the hydrocarbons and mixtures thereof. Furthermore these design models contain water in a total amount—respectively in relation to the total weight of the design model—from 0 to 2.0% by weight.-%. In addition, these embodiments preferably have a weight ratio of the anionic polymer A to the amphoteric polymer B of about 1:0.75. In addition, these design models preferably have a weight ratio of the anionic polymer A to the amphoteric polymer B of about 1:0.75.

By using a special anionic polymer A in combination with a certain amphoteric polymer B, a uniform water-resistant pigment-containing film is formed. This film is extremely stable against outside environmental influences such as water, sweat and abrasion, but can be removed from the keratinous surface without residue by the single use of a surfactant-containing cleaning agent. In addition, the use of this polymer combination leads to a high hold and a high flexibility of the polymer film formed. When these agents are applied to the hair, a high and moisture-resistant hold is also achieved at the same time as a temporary change in color. In addition, these agents have good cosmetic properties after their application to the skin and hair.

In addition to the components described above, the cosmetic agents in the present disclosure can contain further ingredients. The group of these further ingredients includes, in particular, cosmetically active auxiliaries and additives, such as surfactants, care ingredients, thickeners and pH agents.

The agents in the present disclosure can additionally contain at least one nonionic surfactant. Suitable nonionic surfactants are alkyl polyglycosides and alkylene oxide additives with fatty alcohols and fatty acids, each with 2 to 30 moles of ethylene oxide per mole of fatty alcohol or fatty acid. Preparations with good properties are likewise obtained if they contain, as nonionic surfactants, fatty acid esters of ethoxylated glycerol which have been reacted with at least 2 moles of ethylene oxide. The nonionic surfactants are used in a total amount of from about 0.1 to about 45 wt.-%, preferably from about 1 to about 30 wt.-%, most preferably from about 1 to about 15 wt.-% relating to the total weight of the agent.

The agents as contemplated herein can additionally contain at least one cationic surfactant. Cationic surfactants are understood to mean surfactants, i.e. compounds active on the surface, each having one or more positive charges. Cationic surfactants contain only positive charges. These surfactants are usually composed of a hydrophobic part and a hydrophilic head group, where the hydrophobic part generally comprises a hydrocarbon scaffold (for example including one or two linear or branched alkyl chains), and the positive charge (s) are located in the hydrophilic head group. Examples of cationic surfactants are
quaternary ammonium compounds which can carry one or two alkyl chains with a chain length of 8 to 28 carbon atoms as hydrophobic radicals,
quaternary phosphonium salts, substituted with one or more alkyl chains with a chain length of 8 to 28 carbon atoms or
tertiary sulfonium salts.

Furthermore, the cationic charge can also be part of a heterocyclic ring (for example an imidazolium ring or a pyridinium ring) as an onium structure component. In addition to the functional unit that carries the cationic charge, the cationic surfactant can also contain further uncharged functional groups, as is the case, for example, with esterquats. The cationic surfactants are used in a total amount of from about 0.1 to about 45 wt.-%, preferably from about 1 to about 30 wt.-%, most preferably from about 1 to about 15 wt.-%, in relation to the total weight of the agent.

The use of anionic surfactants has been shown to be negative with regard to the abrasion resistance of the pigments on the keratin fibers. For this reason, it is preferred not to use anionic surfactants in the agents in the present disclosure. Anionic surfactants are designated as agents active on the surface with exclusively anionic charges (neutralized by a corresponding counter cation). Examples of anionic surfactants are fatty acids, alkyl sulfates, alkyl ether sulfates and ether carboxylic acids with 12 to 20 carbon atoms in the alkyl group and up to 16 glycol ether groups

In a further preferred design model, agents in the present disclosure are exemplified by the fact that they contain anionic surfactants in a total amount of 0 to about 2.5% wt.-%, preferably from 0 to about 1.5 wt.-%, preferably from 0 to about 0.5 wt.-%, more preferably from 0 to about 0.1 wt.-%, in particular of 0 wt.-%, respectively relating to the total weight of the cosmetic agent.

The agents in the present disclosure can further contain at least one zwitterionic and /or amphoteric surfactant. Suitable zwitterionic surfactants are betaines, N-alkyl-N, N-dimethylammonium glycinate, N-acylaminopropyl-N, N-dimethylammonium glycinate, and 2-alkyl-3-carboxymethyl-3-hydroxyethyl-imidazoline. A preferred zwitterionic surfactant is known under the INCI name Cocamidopropyl Betaine Suitable amphoteric surfactants are N-alkylglycine, N-alkyl propionic acid, N-alkylamino butyric acid, N-alkyliminodipropionic acid, N-hydroxyethyl-N-alkylamidopropylglycine, N-alkyl taurine, N-alkyl sarcosine, 2-alkylaminopropionic acid and alkylamino acetic acid Particularly preferable surfactants are N-cocoalkylaminoproprionate, cocoacylaminoethylaminoproprionate and C₁₂-C₁₈-acylsarcosine.

The amphoteric and/or zwitterionic surfactants are used in a total amount of from about 0.1 to about 45 wt.-%, preferably from about 1 to about 30 wt.-%, most preferably from about 1 to about 15 wt.-%—based on the total weight of the agent.

An advantage of the agents in the present disclosure is that they can be assembled in a variety of forms. When applied with a sponge or a small brush, very even color effects and water and friction-fast coloration can be achieved. However, it is also possible to assemble the agents in the present disclosure as a spray. In particular, the colorations resulting from spray application are also remarkably uniform and water-resistant.

Depending on the chosen form of application, the agents in the present disclosure are set to a certain viscosity. This is usually done by using one or more thickeners. In principle, the choice of thickening agent is unlimited. It is possible to use either organic or purely inorganic thickeners.

Suitable thickening agents are anionic, synthetic polymers; cationic, synthetic polymers; naturally occurring thickeners, such as nonionic guar gums, scleroglucan gums or xanthan gums, gum arabic, ghatti gum, karaya gum, tragacanth gum, carrageenan gum, agar agar, locust bean gum, pectins, alginates, starch fractions and derivatives such as amylose, amylopectin and dextrins, as well as cellulose derivatives, such as, for example, methyl cellulose, carboxyalkyl celluloses and hydroxyalkyl celluloses; nonionic, fully synthetic polymers such as polyvinyl alcohol or polyvinyl pyrrolidinone; as well as inorganic thickeners, in particular layered silicates such as bentonite, especially smectites such as montmorillonite or hectorite.

The viscosity of the agents can be particularly simply and reproducibly set by employing polysaccharides, in particular polysaccharides from the group of carboxy-C₁-C₆-alkyl cellulose, the hydroxy-C₂-C₈-set alkyl celluloses, the alginic acids and/or xanthan gum.

By varying the amount of polysaccharide used, the agent can be assembled both as a gel for brush or sponge application or as a low-viscosity, sprayable solution. The other ingredients of the recipe or the amounts used do not have to be adjusted here, which is particularly of advantage for the production of the agents. In a further particularly preferred design model, an agent in the present disclosure is therefore exemplified by the fact that that it contains in addition at least one polysaccharide from the group of carboxy-C₁-C₆-alkyl cellulose, the hydroxy-C₂-C₈-alkyl celluloses containing alginic acids and/or xanthan gum.

In a most particularly preferred design model, an agent as contemplated herein is exemplified by the fact that it contains in addition at least one polysaccharide from the group of the hydroxy-C₂-C₈-alkyl celluloses as a thickener.

The thickener (s) can be used in the agents in the present disclosure in a total amount of from about 0.1 to about 4.5 wt.-%, preferably from about 0.15 to about 3.5 wt.-% and particularly preferably from about 0.2 to about 2.0 wt.-% relating to the total weight of the agent.

The agents in the present disclosure can contain one or more alkalizing agents to adjust the pH.

The alkalizing agents which can be used in the present disclosure to set the desired pH values can be selected from the group including ammonia, alkanolamines, basic amino acids and inorganic alkalizing agents such as alkali (earth) metal hydroxides, alkali (earth) metal metasilicates, alkali (earth) metal phosphates and alkali (earth) metal hydrogen phosphates. To adjust the pH, the agents in the present disclosure can contain one or more acids. Suitable acids are, for example, organic acids such as alpha-hydroxycarboxylic acids or inorganic acids.

Furthermore, the agents can contain one or more nonionic polymers. Suitable nonionic polymers are for example:
vinyl pyrrolidone/vinyl ester copolymers,
starch and its derivatives, especially starch ether,
Shellac
polyvinyl pyrrolidones.

Furthermore, the agents in the present disclosure can contain further active ingredients, auxiliaries and additives, such as, for example, linear cationic polymers such as quaternized cellulose ethers, polysiloxanes with quaternary groups, dimethylamine-ethyl methacrylate-vinyl pyrrolidinone copolymers quaternized with diethyl sulfate, vinyl pyrrolidinone-imidazolinium-vinyl chloride and methochloride copolymers; zwitterionic and amphoteric polymers (which are different from the polymers of the present disclosure); anionic polymers such as polyacrylic acids or cross-linked polyacrylic acids; structurants such as glucose, maleic acid and lactic acid, hair-conditioning compounds such as phospholipids, for example lecithin and cephalins; perfume oils, dimethyl isosorbide and cyclodextrins; active ingredients that improve fiber structure, in particular mono-, di- and oligosaccharides such as, for example, glucose, galactose, fructose, fruit sugar and lactose; dyes for coloring the agent; anti-dandruff agents such as piroctone olamine, zinc omadine and climbazole; amino acids and oligopeptides; animal and/or vegetable-based protein hydrolyzates, as well as in the form of their fatty acid condensation products or, optionally, anionically or cationically modified derivatives; light stabilizers and UV blockers; active ingredients such as panthenol, pantothenic acid, pantolactone, allantoin, pyrrolidinone carboxylic acids and their salts and bisabolol; polyphenols, in particular hydroxy cinnamic acids, 6,7-dihydroxycumarine, hydroxybenzoic catechins, cannins, leucoanthocyanidins, anthocyanidins, flavanones, flavones and flavanols; ceramides or pseudoceramides; vitamins, provitamins and vitamin precursors; plant extracts; fats and waxes such as fatty alcohols, beeswax, montan wax and paraffins; swelling and penetration substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers and PEG-3 distearate; propellants such as propane-butane mixtures, N₂O, dimethyl ether, CO₂ and air.

Experts will select these further substances according to the desired properties of the agents. With regard to further optional components and the amounts of these components used, reference is expressly made to the relevant manuals known to experts. The additional active ingredients and auxiliaries should preferably be used in the agents in the present disclosure in amounts of from about 0.0001 to about 25 wt.-%, in particular from about 0.0005 to about 15 wt.-%, in relation to the total weight of the cosmetic agent.

When used in the form of a pump spray or of an aerosol spray, the user can spray the agents in the present disclosure directly onto dry hair or skin and thus produce the desired temporary color change. For use on the hair, the user can first style the hair e for example by combing, back-combing, or by using a curling iron, and then spray on the agent It is likewise possible to spray the agent first and to style the hair at the same time or subsequently using the aforementioned methods.

A second subject of the present disclosure is therefore a process for temporarily altering and coloring keratinous fibers, whereby the process comprises the following steps:

a) the provision of a cosmetic agent in the present disclosure in the form of a gel, a pump spray or an aerosol spray,
b) the application, in particular by spray, of the cosmetic agent provided in step a) onto the keratin fibers
c) The distribution of the cosmetic agent applied in step b) onto the keratin fibers and re-shaping the keratin fibers into the desired form.

Keratin-containing fibers are essentially understood to mean all animal hair, e.g. wool, horsehair, angora hair, furs, feathers and products or textiles made from them. However, the keratin fibers should preferably be human hair. With regard to further preferred design models of the method in the present disclosure, in particular with regard to the cosmetic agent used there, the statements regarding the agents in the present disclosure apply mutatis mutandis.

A third subject of the present disclosure is a process for the temporary coloring of the skin, whereby the process comprises the following process steps:

a) the provision of a cosmetic agent in the present disclosure in the form of a gel, pump spray or aerosol,
b) the application, in particular by spray, of the cosmetic agent provided in step a) onto the skin
c) the distribution of the cosmetic agent applied in step b) onto the skin.

With regard to further preferred design models of the method in the present disclosure, in particular with regard to the cosmetic agent used there, the statements regarding the agents in the present disclosure apply mutatis mutandis.

Finally, another subject of the present disclosure is the use of a combination of

a) at least one anionic polymer A, comprising at least one structural unit of the formula (I) and at least one structural unit of the formula (II) and at least one structural unit of the formula (III)

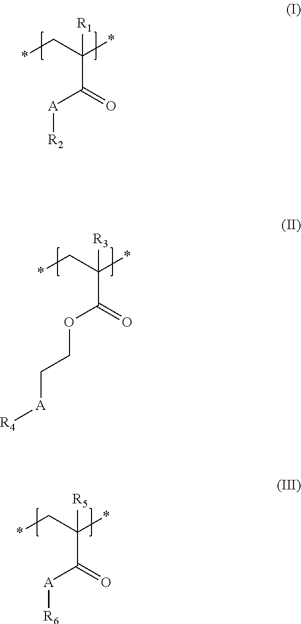

where
R₁, R₃ and R₅, independently of each other, stand for hydrogen or a C₁-C₄-alkyl group,
R₂ stands for a branched saturated C₆-C₁₂-alkyl group,
R₄ stands for a branched saturated C₂-C₅-alkyl group
R₆ stands for hydrogen, a C₁-C₄-alkyl group or a C₂-C₈-hydroxyalkyl group, and
A stands for oxygen, sulfur or an NH group,
b) at least one amphoteric polymer B, comprising at least one structural unit of the formula (IV) and at least one structural unit of the formula (V)

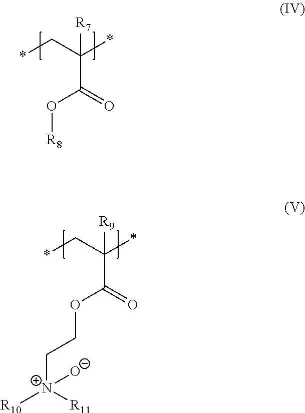

where
R₇ and R₉, independently of each other, stand for hydrogen or a C₁-C₄-alkyl group,
R₈ stands for a linear saturated C₁-C₃₀-alkyl group, and
R₁₀ and R₁₁, independently of each other, stand for a C₁-C₄-alkyl group, and in pigment-containing cosmetic products to improve the water resistance of these products. As contemplated herein, improvement in water resistance means that the use of these polymers leads to a reduction in or complete absence of the pigments deposited on the surface being washed out when water is used.

In a preferred design model of this subject of the present disclosure, the following polymer combination is used:

a) at least one anionic polymer A, comprising at least one structural unit of the formula (I) and at least one structural unit of the formula (II) and at least one structural unit of the formula (III) and at least one structural unit of the formula (IIIa) and at least one structural unit of the formula (IIIb)

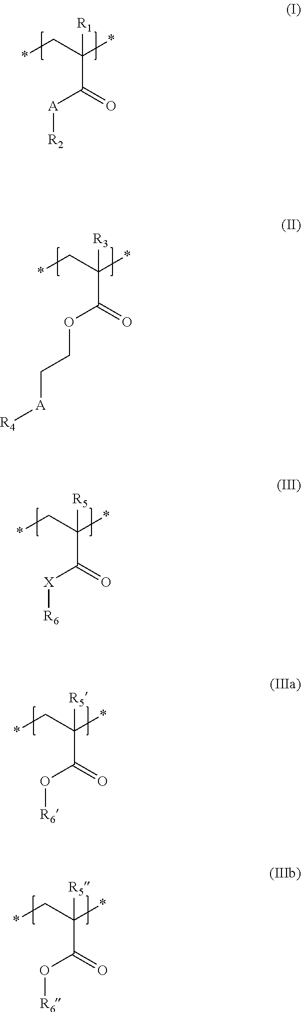

where
R₁ and R₃, each stand for a methyl group,
R₅ and R₆, independently of each other, stand for hydrogen,
R₅′ and R₅″, independently of each other stand for hydrogen or a methyl group, in particular a methyl group,
R₂ stands for a *—C(CH₃)₂—CH₂—C(CH₂)₃-group,
R₄ stands for a *—C(CH₃)₃-group,
A stands for an NH group,
X stands for oxygen,
R₆′ stands for a methyl group and
R₆″ stands for a 2-hydroxypropyl group,
b) at least one amphoteric polymer comprising at least one structural unit of the formula (IV), at least one structural unit of the formula (IVa) and at least one structural unit of the formula (V) contain

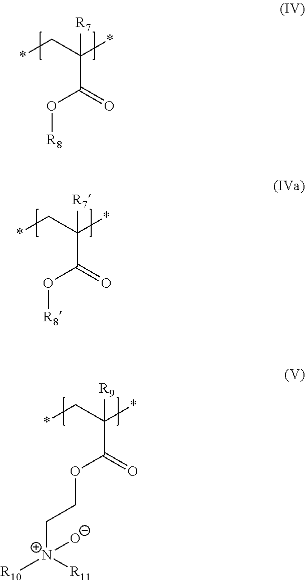

where
R₇, R₇′ and R₈, independently of each other, stand for a methyl group,
R₈ stands for a methyl group,
R₈′ stands for a linear saturated C₁₈-alkyl group and
R₁₀ and R₁₁, independently of each other, stand for a methyl group.

With regard to further preferred design models of the method in the present disclosure, in particular with regard to the polymers used there, the statements regarding the cosmetic agents in the present disclosure apply mutatis mutandis.

The following examples illustrate the present disclosure, without limitation.

### Examples

The following composition was produced:

| Raw material | wt. % |
|---|---|
| Ethanol 99% denat | 79.7 |
| Anionic polymer A ¹⁾ | 0.97 |
| Amphoteric polymer B ²⁾ | 2.5 |
| Pigment ³⁾ | 11 |
| AMP Ultra PC 2000 | 0.09 |
| PVP/VA-copolymer 60/40 W NP | 1.0 |
| Glycerin 99.5% | 2.0 |
| D-Panthenol 75% | 0.20 |
| Dehyquart A CA ⁴⁾ | 0.45 |
| Stearamidopropyl Diemthylamine | 1.3 |
| Perfume | 0.65 |

¹⁾ containing structural units of the formulas (I), (II), (III), (IIIa) and (IIIb) with R₁ and R₃ each = methyl group, R₂ = = *—C(CH₃)₂—CH₂—C(CH₂)₃-group, R₄ = *— C(CH₃)₃-group, R₅ and R₆ each = H, A = NH, X = O, R₅′ and R₅″ each = methyl group, R₆′ = methyl group, R₆″ = 2-hydroxypropyl group,
²⁾ containing structural units of the formulas (IV), (IVa) and (V) with R₇, R₇′ and R₉ each = methyl group, R₈ = methyl group, R₈′ = linear saturated C₁₈-alkyl group R₁₀ and R₁₁ each = methyl group,
³⁾ Colorona Dark Blue, INCI name: MICA, CI 77491 (IRON OXIDES), CI 77891 (TITANIUM DIOXIDE), CI 77510 (FERRIC FERROCYANIDE) (Merck)
³⁾ INCI-name: AQUA (WATER), CETRIMONIUM CHLORIDE (BASF).

The cosmetic agent was obtained by mixing the above ingredients and applied to a strand of hair (Kerling 6-0, light brown). After drying, a uniform dark purple color with a metallic shimmer was obtained. The tress was then washed with water. No influence on the coloring was found. However, the dyeing could be removed once with a residue with a surfactant-containing cleaning composition (shampoo). The use of the special polymer combination therefore leads to a high water resistance of the temporary hair coloring, but without negatively influencing the washability with detergent containing surfactants.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent comprising:
a) at least one anionic polymer A comprising at least one structural unit of the formula (I) and at least one structural unit of the formula (II) and at least one structural unit of the formula (III) 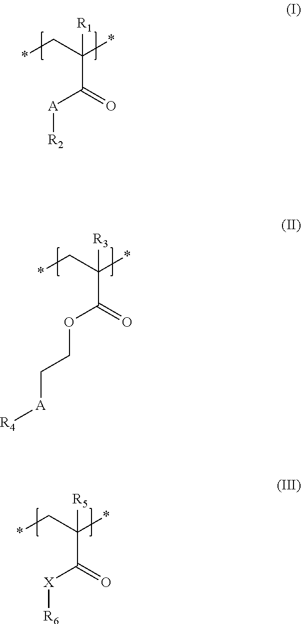
where
R₁, R₃ and R₅, independently of each other, stand for hydrogen or a C₁-C₄-alkyl group,
R₂ stands for a branched saturated C₆-C₁₂-alkyl group,
R₄ stands for a branched saturated C₂-C₅-alkyl group,
R₆ stands for hydrogen, a C₁-C₄-alkyl group or a C₂-C₈-hydroxyalkyl group, and
A and X, independently of each other, stand for oxygen, sulfur or an NH group,
b) at least one amphoteric polymer B comprising at least one structural unit of the formula (IV) and at least one structural unit of the formula (V) 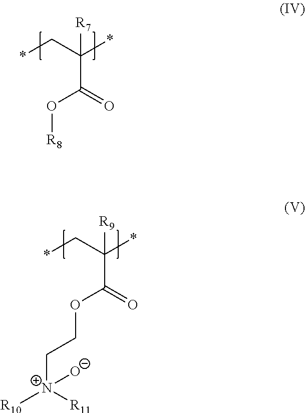
where
R₇ and R₉, independently of each other, stand for hydrogen or a C₁-C₄-alkyl group,
R₈ stands for a linear saturated C₁-C₃₀-alkyl group, and
R₁₀ and R₁₁, independently of each other, stand for a C₁-C₄-alkyl group, and
c) at least one pigment.

2. The cosmetic agent according to claim 1,
characterized by the fact that in the structural unit of the formula (II) the radical R₄ stands for a branched saturated C₂-C₅-alkyl group.

3. The cosmetic agent according to claim 1, characterized by the fact that the minimum one anionic polymer A is included in a total amount of from about 0.1 to about 10 wt.-%, in relation to the total weight of the cosmetic agent.

4. The cosmetic agent according to claim 1, characterized by the fact that in the structural unit of the formula (V) the radicals R₁₀ and R₁₁, independently of each other, stand for a methyl group.

5. The cosmetic agent according to claim 1, characterized by the fact that the at minimum one amphoteric polymer B is included in a total amount of from about 0.1 to about 10 wt.-%, in relation to the total weight of the cosmetic product.

6. The cosmetic agent according to claim 1, characterized by the fact that the minimum one pigment is a colored pigment based on mica or mica and is coated with one or more metal oxides from the group comprising titanium dioxide (CI 77891), black iron oxide (CI 77499), yellow Iron oxide (CI 77492), red and/or brown iron oxide (CI 77491, CI 77499), manganese violet (CI 77742), ultramarine (sodium aluminum sulfasilicate, CI 77007, Pigment Blue 29), chromium oxide hydrate (CI 77289), chromium oxide (CI 77288) and/or iron blue (Ferric Ferrocyanide, CI 77510).

7. The cosmetic composition according to claim 1, characterized by the fact that the minimum one pigment is included in a total amount of from about 1.0 to about 25.0 wt.-%, respectively in relation to the total weight of the cosmetic product.

8. The method of claim 10 further comprising:
a) providing the cosmetic composition in the form of a gel, a pump spray or an aerosol spray,
b) applying the cosmetic composition onto keratin fibers,
c) distributing the cosmetic composition onto the keratin fibers and re-shaping the keratin fibers into the desired form, wherein the cosmetic composition colors the keratin fibers.

9. The method of claim 10 further comprising:
a) providing the cosmetic composition in the form of a gel, a pump spray or an aerosol spray,
b) applying the cosmetic composition onto the skin,
c) distributing the cosmetic composition onto the skin to color the skin.

10. A method for improving water resistance of a pigment-containing cosmetic composition, the method comprising:
adding to the pigment-containing cosmetic composition a cosmetic agent comprising:
a) at least one anionic polymer A, comprising at least one structural unit of the formula (I) and at least one structural unit of the formula (II) and at least one structural unit of the formula (III) 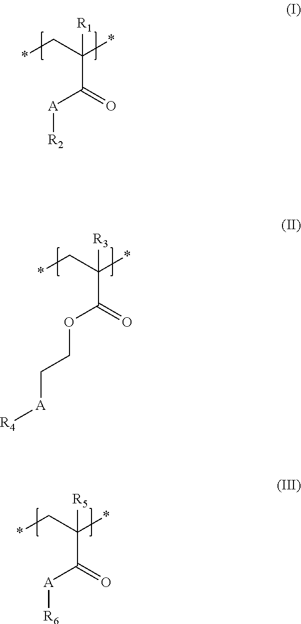
where
R₁, R₃ and R₅, independently of each other, stand for hydrogen or a C₁-C₄-alkyl group,
R₂ stands for a branched saturated C₆-C₁₂-alkyl group,
R₄ stands for a branched saturated C₂-C₅-alkyl group,
R₆ stands for hydrogen, a C₁-C₄-alkyl group or a C₂-C₈-hydroxyalkyl group, and
A stands for oxygen, sulfur or an NH group,
b) at least one amphoteric polymer B, comprising at least one structural unit of the formula (IV) and at least one structural unit of the formula (V) 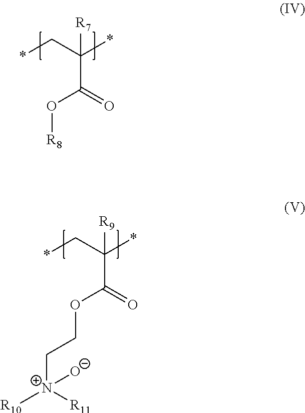
where
R₇ and R₉, independently of each other, stand for hydrogen or a C₁-C₄-alkyl group,
R₈ stands for a linear saturated C₁-C₃₀-alkyl group, and
R₁₀ and R₁₁, independently of each other, stand for a C₁-C₄-alkyl group.
